(19) United States
(12) Patent Application Publication
(10) Pub. No.: US 2020/0363219 A1
(43) Pub. Date: Nov. 19, 2020

(54) TRANSPORTATION ROUTE PLANNING WITH INTEGRATED PERSONAL PHYSICAL ACTIVITY GOALS

(71) Applicant: JONATHAN RABINOWITZ, RAANANA (IL)

(72) Inventor: JONATHAN RABINOWITZ, RAANANA (IL)

(21) Appl. No.: 15/930,729

(22) Filed: May 13, 2020

Related U.S. Application Data
(60) Provisional application No. 62/846,795, filed on May 13, 2019.

(51) Int. Cl. G01C 21/34; A63B 24/00

(52) U.S. Cl. CPC G01C 21/3423; G16H 20/30; A63B 24/0075

(57) ABSTRACT

Integrating attaining personal physical activity goals in transportation route planning, where a user inputs their physical activity goals and their travel destination or destinations, which information is processed and used to optimize personal transportation route planning to get to destinations while attaining the user's physical activity goals.

### BACKGROUND

Exercise is essential to a healthy lifestyle. Increasing physical activity has been identified as a proven way to improve health and is widely encouraged and promoted by the health care community. People know they should be active, but they are not able to integrate it into their lives. Most people travel from their homes to go to work, school or to pursue other daily activities. What is needed, then, are methods, systems, and products for integrating physical activity in daily routines such as travel.

### SUMMARY

The invention, in embodiments thereof, provides for integrating physical activity goals in personal transportation route planning.

Systems, methods, and products according to the exemplary embodiments contained herein, will be apparent to one with ordinary skill in the art. It is intended that all such additional systems, methods, and/or computer program products included within this description, be within the scope of the claims, and be protected by the accompanying claims.

### BRIEF DESCRIPTION OF THE DRAWINGS

The invention will be better understood when the following Detailed Description is read with reference to the accompanying drawings, wherein:

FIG. 1 is a simplified conceptual diagram of a travel and exercise planning system, constructed and operative in accordance with an embodiment of the invention;

FIG. 2 is a flow chart of user experience, useful in illustrating an embodiment of the invention; and

FIG. 3 are sample output displays, useful in illustrating an embodiment of the invention.

### DETAILED DESCRIPTION

The invention, in exemplary embodiments thereof, will now be described more fully hereinafter with reference to the accompanying drawings. The exemplary embodiments may, however, be embodied in many different forms and should not be construed as limited to the embodiments set forth herein. These embodiments are provided so that this disclosure will be thorough and complete and will fully convey the exemplary embodiments to those of ordinary skill in the art. Moreover, all statements herein reciting embodiments, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those of ordinary skill in the art that the diagrams, schematics, illustrations, and the like represent conceptual views or processes illustrating the exemplary embodiments. The functions of the various elements shown in the figures may be provided through the use of dedicated hardware as well as hardware capable of executing associated software. Those of ordinary skill in the art further understand that the exemplary hardware, software, processes, methods, and/or operating systems described herein are for illustrative purposes and, thus, are not intended to be limited to any particular named manufacturer.

As used herein, the singular forms “a,” “an,” and “the” are intended to include the plural forms as well, unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof.

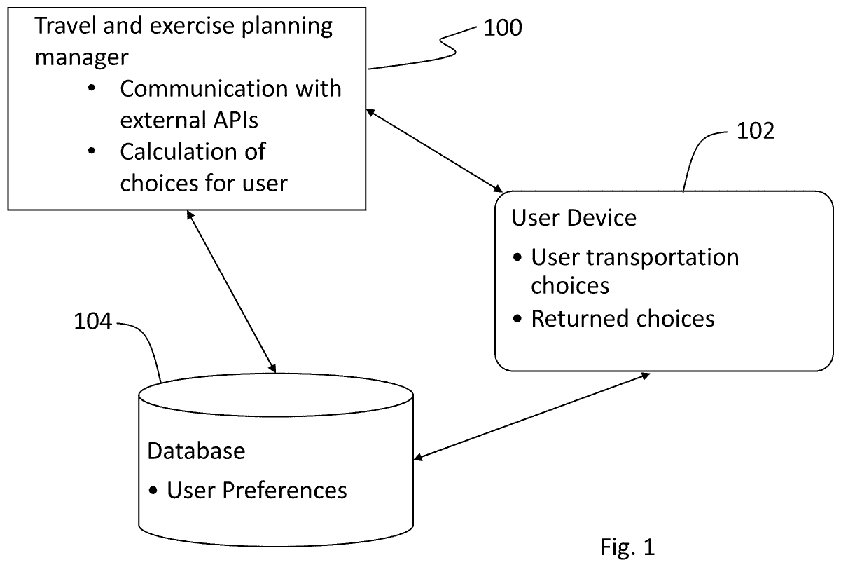

As seen in FIG. 1, a travel and exercise planning manager 100 uses data from a user's device 102. Examples of user device 102 include cellphones and smartwatches. User device 102 provides data on the user's transportation choices, such as their destination and location. Data for travel and processing manager 100 is stored in database 104. Travel and exercise planning manager 100 and database 104 can be located on user device 102 or on a remote server.

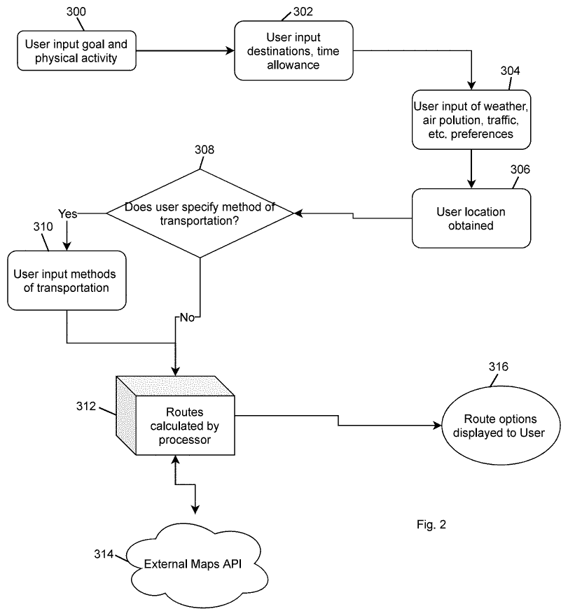

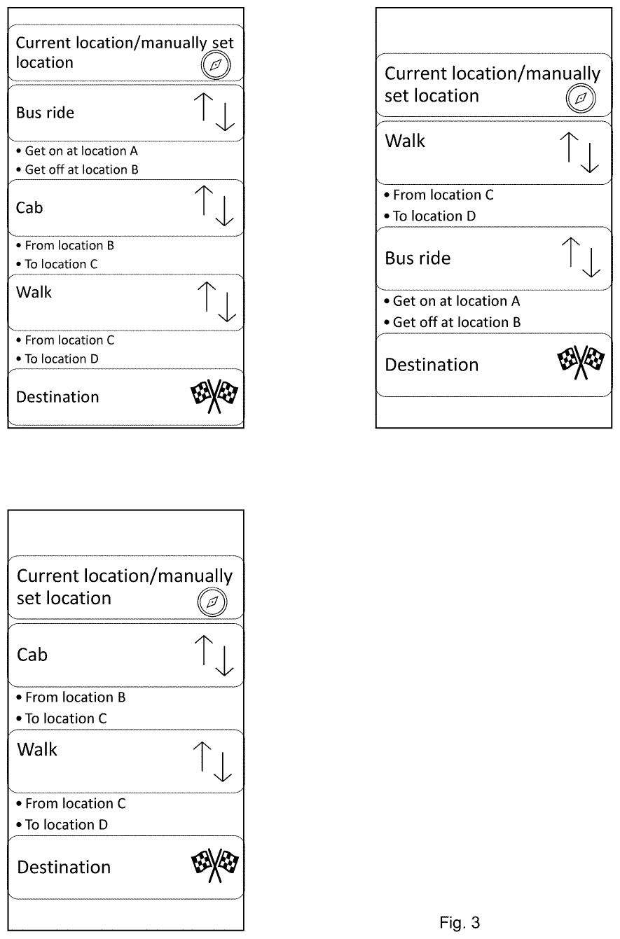

Additional reference is now made to FIG. 2, which is a flow chart illustrating a user's experience in using the system of FIG. 1. The user inputs their physical activity goal (e.g., walk 2 miles a day, burn 1000 calories walking, bike 10 miles) (step 300). Next, the user inputs their destinations, for example their place of work, and when they would like to leave and when they would like to arrive (step 302). The user is given the option (step 304) to specify whether other variables should be taken into consideration in route planning such as preferred weather conditions, or aversion to air pollution, traffic or congestion of travel methods (e.g., avoid crowded subway lines). The user's current location is obtained (step 306) either from the GPS on the user's device or can be manually input by the user. The user is given the option to specify methods of transportation. If the user opts to specify methods of transportation (step 308), they specify their choices in step 310, for example the user might specify to travel only by bus or only by private automobile. The route is calculated by processor (step 312) using data from external maps (step 314). The calculation (step 314) is done by selectively adjusting various limiters and minimums (e.g., tolerance for walking to next stop) in known available mapping algorithms to obtain the desired results. The route is displayed to the user (step 316) (See FIG. 3, Example screen of output). The travel and exercise manager also tracks the users daily physical activity and saves in the database. The travel and exercise manager can also work alongside other physical activity trackers.

The following use case is provided by way of example of one of the possible embodiments. A user, Jack, spends an hour a day commuting to work. He is struggling to make time for physical activity. Jack sets a goal of walking 10 miles a week. He defines his anticipated commuting times and destinations. He works Monday to Friday. Travel and exercise planning manger 100 arranges his transportation so that he walks 10 miles during the week. It optimizes transportation route, while taking into account weather conditions (e.g., if rain is forecast for the afternoon incorporates walk in the morning commute). For example, on Monday morning since it is expected to rain in the afternoon, he is directed to take the 32 bus that comes at 8:15 which has a stop 2 miles away, further away than the bus stop closer to his home. It also gives him the walking route to that bus and from that bus to work. On Tuesday, since he indicated a preference to walk in the neighborhood near where he works, he is directed to take an express bus next to his home that has a stop 1 mile from his place of work, and to also take that bus home from work.

Any aspect of the invention described herein may be implemented in computer hardware and/or computer software embodied in a non-transitory, computer-readable medium in accordance with conventional techniques, the computer hardware including one or more computer processors, computer memories, I/O devices, and network interfaces that interoperate in accordance with conventional techniques.

It is to be appreciated that the term “processor” or “device” as used herein is intended to include any processing device, such as, for example, one that includes a CPU (central processing unit) and/or other processing circuitry. It is also to be understood that the term “processor” or “device” may refer to more than one processing device and that various elements associated with a processing device may be shared by other processing devices.

The term “memory” as used herein is intended to include memory associated with a processor or CPU, such as, for example, RAM, ROM, a fixed memory device (e.g., hard drive), a removable memory device (e.g., diskette), flash memory, etc. Such memory may be considered a computer readable storage medium.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

Embodiments of the invention may include a system, a method, and/or a computer program product. The computer program product may include a computer readable storage medium (or media) having computer readable program instructions thereon for causing a processor to carry out aspects of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the invention.

Aspects of the invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart illustrations and block diagrams in the drawing figures illustrate the architecture, functionality, and operation of possible implementations of systems, methods, and computer program products according to various embodiments of the invention. In this regard, each block in the flowchart illustrations or block diagrams may represent a module, segment, or portion of computer instructions, which comprises one or more executable computer instructions for implementing the specified logical function(s). In some alternative implementations, the functions noted in a block may occur out of the order noted in the drawing figures. For example, two blocks shown in succession may, in fact, be executed substantially concurrently, or the blocks may sometimes be executed in the reverse order, depending upon the functionality involved. It will also be noted that each block of the flowchart illustrations and block diagrams, and combinations of such blocks, can be implemented by special-purpose hardware-based and/or software-based systems that perform the specified functions or acts.

The descriptions of the various embodiments of the invention have been presented for purposes of illustration, but are not intended to be exhaustive or limited to the embodiments disclosed. For example, the systems and methods described herein are applicable to any type of structure on semiconductor wafers. Many modifications and variations will be apparent to those of ordinary skill in the art without departing from the scope and spirit of the described embodiments.

1-2. (canceled)

3. A travel and exercise planning device comprising:
an input interface adapted to receive input from a user;
an output interface adapted to provide output to a user;
a processor; and
a memory device storing computer software code, the code when executed causing the processor to perform operations, the operations comprising
receiving, via said input interface, inputs from a user indicating a desired physical activity goal,
obtaining at least one travel origin and at least one corresponding travel destination for the user,
computing at least one travel route, from said at least one travel origin to said at least one corresponding travel destination, said at least one travel route having at least one physical activity task associated therewith, said at least one physical activity task being configured to enable said user to achieve said desired physical activity goal by performing said at least one physical activity task while traversing said at least one travel route, and
providing to said user, via said output interface, said at least one travel route and said at least one physical activity task associated with said at least one travel route.

4. A travel and exercise planning system associated with a user device via a communication network, the user device including an input interface and an output interface, the system comprising:
a processor; and
a memory device storing computer software code, the code when executed causing the processor to perform operations, the operations comprising
receiving from the user device inputs indicating a desired physical activity goal of the user,
obtaining at least one travel origin and at least one corresponding travel destination for the user,
computing at least one travel route, from said at least one travel origin to said at least one corresponding travel destination, said at least one travel route having at least one physical activity task associated therewith, said at least one physical activity task being configured to enable said user to achieve said desired physical activity goal by performing said at least one physical activity task while traversing said at least one travel route, and
outputting said at least one travel route and said at least one physical activity task associated therewith, via the communication network, to the user device for display on the output interface thereof.

5. The travel and exercise planning device of claim 3, wherein:
said desired physical activity goal comprises a long-term goal;
said obtaining said at least one travel origin and said at least one corresponding travel destination comprises obtaining a plurality of pairs of said travel origins and corresponding travel destinations; and
said computing said at least one travel route comprises computing, for each said pair of travel origin and corresponding travel destination, a said travel route, wherein a cumulative physical activity component of all said physical activity tasks associated with all said travel routes together meets said desired physical activity goal.

6. The travel and exercise planning device of claim 3, wherein said obtaining said at least one travel origin comprises obtaining said at least one travel origin from a Global Positioning System, based on a current location of said system.

7. The travel and exercise planning device of claim 3, wherein said operations further include receiving, via said input interface, inputs from the user indicating user preferences with respect to at least one of weather, air pollution, traffic, type of physical activity, time of physical activity, and location of physical activity, and
wherein said computing said at least one travel route includes taking into consideration, during said computing, said user preferences.

8. The travel and exercise planning device of claim 7, wherein:
said operations further include obtaining, from an external system, additional information associated with said user preferences; and
said taking into consideration includes taking said additional information into consideration during said computing.

9. The travel and exercise planning device of claim 8, wherein said additional information includes at least one of weather information, traffic information, public transport schedule information, public transport crowding information, and air quality information.

10. The travel and exercise planning device of claim 3, said operations further comprising at least one of:
storing, in a memory component associated with said device, information relating to at least one of said at least one physical activity task associated with said at least one travel route and user completion of said physical activity task over time; and
transmitting information relating to said at least one physical activity task and/or completion thereof to an external exercise tracking system.

11. The travel and exercise planning device of claim 3, wherein said desired physical activity goal is a goal indicating a minimum threshold of desired physical activity per time unit.

12. The travel and exercise planning system of claim 4, wherein:
said desired physical activity goal comprises a long-term goal;
said obtaining said at least one travel origin and said at least one corresponding travel destination comprises obtaining a plurality of pairs of said travel origins and corresponding travel destinations; and
said computing said at least one travel route comprises computing, for each said pair of travel origin and corresponding travel destination, a said travel route, wherein a cumulative physical activity component of all said physical activity tasks associated with all said travel routes together meets said desired physical activity goal.

13. The travel and exercise planning system of claim 4, wherein said operations further include receiving, via said input interface, inputs from the user indicating user preferences with respect to at least one of weather, air pollution, traffic, type of physical activity, time of physical activity, and location of physical activity, and
wherein said computing said at least one travel route includes taking into consideration, during said computing, said user preferences.

14. The travel and exercise planning system of claim 12, wherein:
said operations further include obtaining, from an external system, additional information associated with said user preferences; and
said taking into consideration includes taking said additional information into consideration during said computing.

15. A method for travel and exercise planning for a user, the user associated with a user device including an input interface and an output interface, the method comprising:
receiving, via said input interface, inputs from a user indicating a desired physical activity goal;
obtaining at least one travel origin and at least one corresponding travel destination for the user;
computing at least one travel route, from said at least one travel origin to said at least one corresponding travel destination, said at least one travel route including at least one physical activity task, said at least one physical activity task being configured to enable said user to achieve said desired physical activity goal by performing said at least one physical activity task while traversing said at least one travel route; and
providing to the user, via said output interface, said at least one travel route and said at least one physical activity task associated with said at least one travel route.

16. The method of claim 15, wherein:
said desired physical activity goal comprises a long-term goal;
said obtaining said at least one travel origin and said at least one corresponding travel destination comprises obtaining a plurality of pairs of said travel origins and corresponding travel destinations; and
said computing said at least one travel route comprises computing, for each said pair of travel origin and corresponding travel destination, a said travel route, wherein a cumulative physical activity component of all said physical activity tasks associated with all said travel routes together meets said desired physical activity goal.

17. The method of claim 15, wherein said obtaining said at least one travel origin comprises obtaining said at least one travel origin from a Global Positioning System, based on a current location of said system.

18. The method of claim 15, further including receiving, via said input interface, inputs from the user indicating user preferences with respect to at least one of weather, air pollution, traffic, type of physical activity, time of physical activity, and location of physical activity, and
wherein said computing said at least one travel route includes taking into consideration, during said computing, said user preferences.

19. The method of claim 18, further including obtaining, from an external system, additional information associated with said user preferences and wherein said taking into consideration includes taking said additional information into consideration during said computing.

20. The method of claim 19, wherein said additional information includes at least one of weather information, traffic information, public transport schedule information, public transport crowding information, and air quality information.

21. The method of claim 15, further comprising at least one of:
storing, in a memory component associated with said user device, information relating to at least one of said at least one physical activity task associated with said at least one travel route and user completion of said physical activity task over time; and
transmitting information relating to said at least one physical activity task and/or completion thereof to an external exercise tracking system.

22. The method of claim 3, wherein said desired physical activity goal is a goal indicating a minimum threshold of desired physical activity per time unit.
